(19) United States
(12) Patent Application Publication
(10) Pub. No.: US 2026/0325726 A1
(43) Pub. Date: Oct. 22, 2026

(54) BREAST MILK EXTRACTION AND STORAGE BAG DEVICES AND SYSTEMS

(71) Applicant: MOMment LLC, Maple Grove, MN (US)

(72) Inventors: Kayla Marie Backes, Maple Grove, MN (US); Carley Childers, Saint Joseph, MN (US); Scott Melanson, Maple Grove, MN (US)

(73) Assignee: MOMment LLC, Maple Grove, MN (US)

(21) Appl. No.: 19/654,816

(22) Filed: Apr. 22, 2026

Related U.S. Application Data
(60) Provisional application No. 63/792,782, filed on Apr. 22, 2025.

(51) Int. Cl. A61M 1/06

(52) U.S. Cl. CPC A61M 1/064; A61M 1/062; A61M 2202/0014; A61M 2205/128

(57) ABSTRACT

Devices, systems and methods for breast milk expression and storage including a storage reservoir configured to store the breast milk after expression, a funnel portion connected directly or indirectly to the storage reservoir, a flexible diaphragm configured to transmit externally applied vacuum pulses into the interior air space of the funnel portion as suction, the flexible diaphragm connected to an outer wall of the device in a liquid tight manner, and a valve configured to allow breast milk flow into the storage reservoir and prevent backflow of breast milk out of the storage reservoir. The funnel portion, the flexible diaphragm, and the valve may be fully integrated as a single device, which may function as a disposable liner, eliminating the need to wash breast pump parts after use.

### PRIORITY CLAIM

This application claims priority to U.S. Provisional application number 63/792,782, filed Apr. 22, 2025, entitled Breast Pump System with Liner Collection Bag, the disclosure of which is hereby incorporated by reference in the entirety.

### BACKGROUND

Pumps are common devices which are used to assist lactating individuals in extracting milk, such as when they are separated from their children for extended periods of time or if breastfeeding isn't an option or it isn't the preferred method of providing breastmilk to their children. They may be commonly used if a lactating person is exclusively pumping or when a breast feeding person goes to, or returns to work, travels outside of the home or other times of extended separation from the child when the lactating person wishes to continue to provide the benefits of breast milk, as well as to continue breast feeding at those times when home with the child. Breast pumps, therefore, provide tremendous advantages by providing lactating people the opportunity to provide their children with breastmilk even if breastfeeding isn't an option or isn't the preferred option and allows those who are breastfeeding time apart from their children.

Despite the benefits of breast pumps, the process of pumping milk is time consuming and is made more difficult due to factors such as large and rigid breast pumping equipment that isn't easily portable, is bulky, and obstructs movements. In addition, breast pumps typically include multiple components which must all be carefully washed, dried, and stored after each use. This is to prevent bacterial or fungal growth within the components, due to the presence of residue milk, which could linger in the pieces and could then contaminate milk during subsequent collections.

Although frequent washing of the breast pump components is necessary, lactating people may not have access to clean and private places to wash the components or to set them down during the washing process while carefully washing each separate piece. For example, public restrooms and hotel bathrooms often have sink basins with little or no places to hygienically place the washed components while proceeding through the cleaning process. For those who wait to clean parts at home, it requires the burden of carrying dirty parts around which isn't reasonable while traveling. In addition to being difficult, the washing process adds a substantial amount of additional time to the breast pumping process, which can be problematic while working or traveling. It also may cause the users to worry that the washing process was not adequately performed due to the difficulty of the process, time restraints, and/or lack of a clean and hygienic space. The stress of the process and the time lost from work may be significant factors which cause people to give up breast feeding. A better breast pump system is needed to help breast feeding individuals continue to provide the benefits of breast milk to their children.

### SUMMARY

Various embodiments include device for breast milk expression and storage including a storage reservoir configured to store the breast milk after expression; a funnel portion comprising one or more outer walls forming a tubular shape which tapers to a more narrow diameter as it extends toward a front end and an interior space within the one or more walls, the funnel portion connected directly or indirectly to the storage reservoir; a flexible diaphragm, wherein the flexible diaphragm is configured to transmit externally applied vacuum pulses into the interior air space of the funnel portion as suction, wherein the flexible diaphragm is connected to an outer wall of the device in a liquid tight manner; and a valve configured to allow breast milk flow into the storage reservoir and prevent backflow of breast milk out of the storage reservoir. The storage reservoir, the funnel portion, the flexible diaphragm, and the valve may be fully integrated as a single device.

The device may further include an upper portion connecting the funnel portion and the storage reservoir. The flexible diaphragm may be located in a wall of the upper portion. A valve may be located between the upper portion and the storage reservoir, such that breast milk located in the upper portion flows through the valve and into the storage reservoir by gravity. The upper portion and the storage reservoir may be releasably connected by a connection feature to allow separation of the reservoir portion from the upper portion. The connection feature may be configured to allow liquid tight closure of the reservoir portion after separation of the reservoir portion.

In some embodiments, the funnel portion may be a flexible liner configured to line an interior wall of a separate more rigid external funnel, wherein the funnel portion prevents contact between the breast milk and the external funnel. The flexible diaphragm may be within the funnel portion in some embodiments, or may be located within and integrated with the storage reservoir in some other embodiments.

In some embodiments, the device may also include a ring surrounding the flexible diaphragm and a cap connected to or configured to connect to the ring on an exterior aspect of the system and forming an airspace between the flexible diaphragm and the cap, the cap further configured to connect to a vacuum tube.

Various embodiments include a beast milk expression and storage bag device including a funnel portion including one or more walls forming a tubular shape having an open rear end and tapering inward as it extends toward a front end, the funnel portion configured to surround an interior space, an upper portion including one or more outer walls comprising flexible sheets configured to surround an interior space, a flexible diaphragm integrated with the one or more outer walls of the upper portion, and an aperture in the one or more outer walls of the upper portion, the front end of the funnel portion connected to the upper portion at the aperture of the upper portion to provide a liquid tight connection between the interior space of the funnel portion and the interior space of the upper portion. The device may further include a storage reservoir connected to the upper portion including one or more walls comprising flexible sheets configured to surround an interior space, and a valve configured to provide a sole flow path for one direction flow of breast milk from the interior space of the upper portion to the interior space of the lower portion. The storage reservoir may be located below the upper portion. The valve may be located at or formed by a lower edge of one or more of the one or more walls of the upper portion. The device may also include a sealable closure providing liquid tight enclosure of the storage reservoir. The upper portion and the storage reservoir may be releasably connected to each other by the sealable closure. The device of claim may further include a ring surrounding the flexible diaphragm, the ring being of a more rigid material than a material of the flexible diaphragm. The flexible diaphragm may be the same material as a material of the one or more outer walls with which it is integrated.

Various embodiments include a beast milk expression and storage bag device including a storage reservoir configured to store the breast milk after expression, a funnel portion comprising one or more walls forming a tubular shape having an open rear end and tapering inward as it extends toward a front end and an interior space within the one or more walls, the front end of the funnel portion connected directly or indirectly to the storage reservoir, a valve configured to provide unidirectional flow of breast milk into the storage reservoir, a flexible diaphragm, wherein the flexible diaphragm is configured to transmit externally applied vacuum pulses into the interior air space of the funnel portion as suction, wherein the flexible diaphragm is connected to the system in a liquid tight manner, a ring surrounding the diaphragm, the ring comprising a material which is more rigid than a material of the diaphragm, and a dome shaped cap connected to the ring and providing an airspace within the housing and abutting the flexible diaphragm, the cap comprising a connector configured to connect with a vacuum tube. The dome shaped cap may be releasably connected to the ring and reusable after disconnection.

The device as described above may be a thin-walled structure that can be deformed under manual pressure. The device may include an outer shell as a part of a system, wherein the outer shell comprises a front wall, a rear wall and a space between the front and rear walls, wherein the device is sized and shaped to fit within the outer shell space when empty and when filled with milk. The shell may include a funnel. The funnel may be sufficiently rigid to not collapse upon suction from vacuum pressure applied through the device by an externally applied vacuum source and/or to ensure stability during diaphragmatic oscillation. There may be an opening in the shell for insertion of the device into the shell. There may be an aperture in the shell to facilitate connection of the diaphragm cap to the device and a vacuum source through a wall of the shell. The outer shell may be configured to be releasably coupled to one or more outer walls of the funnel liner device. The shell may include structural rings which may be configured to releasable couple with the funnel device liner to prevent the funnel portion from collapsing upon suction. In some embodiments, the shell may be configured such that the funnel may be repeatedly attached to and removed from the shell as desired by the user. The shell may be sized and shaped to be positioned at least partially beneath the breast during use, displacing breast tissue upward, such as fitting within a cup of a bra. In some embodiments, the shell may have a crescent shape which widest at the center of the shape and tapers thinner towards the ends.

Various embodiments include methods of expressing and collecting breast milk including any of the embodiments described above.

Various embodiments include methods of expressing and collecting breast milk that eliminates the need to clean pump parts after a pumping session including connecting a single-use disposable breast pump part set comprising a funnel, a valve, a diaphragm, and a milk reservoir to a breast pump, abutting the rear end of the funnel portion to the breast while ensuring the nipple is inserted into the interior space of the funnel portion, operating the breast pump to create diaphragmatic oscillation of the diaphragm to extract milk from the nipple that flows into the funnel, through the valve, and down into the milk reservoir, completing the pumping session, and then, removing the single-use pump part set from the breast and nipple, disconnecting the pump part set from the breast pump, and optionally disposing of the pump part set after use, eliminating the necessity of cleaning parts after using them. The valve, diaphragm, funnel, and milk reservoir may be a single-use liner bag device. Disposing of the bag device may optionally happen after the milk has been removed from the storage reservoir, storing the milk in the storage reservoir and removal of the milk from the storage reservoir prior to disposal of the bag device. The storage reservoir may have been separated from the bag device above the sealed section of the storage reservoir. The storage reservoir may have been sealed and separated from the bag device, and the bag device less the storage reservoir may then disposed. The storage reservoir may be disposed of after the removal of milk from the storage reservoir. The storage reservoir may be stored prior to removal of milk from the storage reservoir and subsequent disposal of said storage reservoir. The device may be connected directly to a breast pump. The bag device may be indirectly connected to a breast pump via vacuum tubing. The bag device may be indirectly connected to a breast pump via an accessory of the breast pump to oscillate the diaphragm mechanically. The pump part set may be a thin-walled structures that can be deformed by manual pressure. The valve, diaphragm, funnel, and milk reservoir may be a liner bag device that lines an external more rigid funnel which it is also coupled to for funnel structure and to prevent the funnel from collapsing inward upon suction during use.

### BRIEF DESCRIPTION OF THE DRAWINGS

The following drawings are illustrative of particular embodiments and do not limit the scope of the inventions. The drawings are not necessarily to scale and are intended for use in conjunction with the following detailed description. Embodiments of the inventions will be described with reference to the drawings, in which like numerals may represent like elements.

FIG. 1 is a front perspective view of the breast milk expression and storage bag device;

FIG. 2 is a front perspective view of the breast milk expression and storage bag device of FIG. 1 with the storage reservoir detached;

FIG. 3 is a rear perspective view of the shell in a use configuration;

FIG. 4 is a rear perspective view of the shell of FIG. 3 in a storage configuration;

FIG. 5 is a front perspective view of the shell of FIG. 3 and the vacuum cap and vacuum tube, both detached;

FIG. 6 is a cross sectional view of the breast milk expression and storage bag device and shell along with the vacuum cap and vacuum tube, both detached;

FIG. 7 is cross sectional view of the breast milk expression and storage bag device and shell of FIG. 6 with the vacuum cap attached and the vacuum tube detached;

FIG. 8 is a cross sectional view of the breast milk expression and storage bag device and shell of FIG. 6 with the vacuum cap and vacuum tube attached;

FIG. 9 is a top view an example of a single chamber device according to various embodiments;

FIG. 10 is a side perspective view of the device of FIG. 9;

FIG. 11 is a closeup view of the diaphragm portion of the device according to various embodiments;

FIG. 12 is a closeup view of the diaphragm portion of the device according to various alternative embodiments; and

FIG. 13 is a closeup view of the diaphragm portion of the device according to various other alternative embodiments.

### DETAILED DESCRIPTION

The following detailed description is exemplary in nature and is not intended to limit the scope, applicability, or configuration of the inventions. Rather, the following description provides practical illustrations for implementing various exemplary embodiments. Utilizing the teachings provided herein, those skilled in the art may recognize that many of the examples have suitable alternatives that may be utilized. In the following description, the orientations are described from the perspective of the user. As such, the side of the system that faces the user is described as the rear, while the opposing side (facing away from the user) is described as the front. This is for ease of understanding only. In addition, the figures provided herein show devices for use on a right breast. It should be understood that the breast milk collection system may also include devices for use on the left breast, and such devices may be mirror image of the right breast milk devices shown herein. In still other embodiments, the devices may be shaped such that they may be used on either side, and the breast milk collection system may therefore include pairs of two identical devices.

The invention described herein includes a breast milk expression and storage liner bag device that connects to the breast pump and forms the only surface in contact with the milk during pumping. The device includes a storage reservoir to store the breast milk after expression, and a flexible diaphragm, contiguous with and integrated into the device (though in alternative embodiments one or more components such as the flexible diaphragm may be removably connected to the device). The flexible diaphragm is configured to transmit externally applied vacuum pulses (or to be mechanically manipulated to flex and oscillate and create vacuum pulses within the device) into the interior air space of the device and may form a portion of the wall of the device and/or be affixed to the wall of the device in a liquid tight manner. The device may also include a valve to prevent backflow of breast milk out of the storage reservoir while still providing a path for milk to enter the storage reservoir. It may also include a funnel portion, which may be directly or indirectly connected to the storage reservoir. Various locations of the flexible diaphragm and the valve are possible. A cap may connect to and cover the flexible diaphragm, such as by a cap surrounding the diaphragm, to transmit vacuum pulses (such as from an external vacuum source or from direct manipulation of the diaphragm itself) to the interior space of the device. The integrated design of the flexible diaphragm prevents the possibility of liquid or air flow through the valve. The device may be made entirely of flexible material and may be shaped to use as a liner bag for an external support device which may be or may include a more rigid funnel, such that milk only contacts the interior space of the device. For example, the flexible sheet material may be thin walled, such as less than about 0.10 inches, or less than about 0.050 inches, or less than about 0.040 inches, such as between about 0.005 and about 0.050 inches, or about 0.010 to about 0.030 inches, or about 0.010 inches to 0.020 inches. The material may be easily deformable, foldable and/or collapsable with manual pressure. The material may further resist cracking when freezing. The entire device, except optionally the diaphragm and/or diaphragm ring, may be made from such thin film material, which may be the same material throughout or different materials. Alternatively, the device may be used alone, without any external support, or with only a partial external support such as only an exterior funnel. In such embodiments, the device may still be made of a flexible sheet material which is thin walled, and/or collapsable with manual pressure, though it may optionally be slightly thicker and stronger than the liner embodiments as it must be self supporting. In various embodiments, the liner device and/or the self supporting device may be made entirely of the thin flexible sheet material, including the valve, the funnel portion, and the diaphragm. In various other embodiments, the liner device and/or the self supporting device may be made entirely of the thin, flexible sheet material except for small portions of the device which may be more rigid, such as the diaphragm ring and/or the diaphragm and/or the funnel portion. However, in such embodiments, the device may nevertheless be provided to users in a flat configuration, with the portions of the sheet folded to lie flat against each other, and the diaphragm ring and/or diaphragm also lying parallel with the sheets, so that they may be easily stored and stacked. Because of the thin flexible film used in construction and the low profile of the diaphragm rim, the device may be thin when folded into a flat package. The device may be folded 2 or three or four or five times within a packet or for sale within a package or for storage or transport, for example. For example, the folded device may be less than about 1 inch, or less than about 0.5 inches, or less than about 0.3 inches, and greater than about 0.05 or 0.01 inches in maximum thickness when folded flat. For example, the folded device may have a maximum thickness about 0.05 and about 1 inch, or between about 0.05 or about 0.01 and 0.5 inches, or between about 0.05 and about 0.2 inches.

In various embodiments, the breast milk expression and storage bag device acts as an independent expression and collection and storage device, or as a liner device within a support, that connects to the breast pump and forms the only surface in contact with the milk during pumping. The breast milk expression and storage bag device may be contained within an optional shell which also does not contact milk during pumping. As a result, milk can be collected multiple times, without the need to wash or sterilize the breast pump system or its components such as the diaphragm or valve or the shell between collection. When a pumping session is complete, the breast milk expression and storage bag device can be removed from the breast pump system and from the optional shell or other supports and used for milk storage, and in some embodiments may be used for feeding as well. The breast milk expression and storage liner bag may discarded after a single use. A new breast milk expression and storage bag device can be connected to the breast pump system for milk collection during a subsequent pumping session, without the need to wash or sterilize any components of the breast milk expression and storage bag device or the optional shell or other external supports or components of the breast pump system. This saves a substantial amount of time and inconvenience and removes a significant obstacle for breast feeding persons while significantly improving the steps to breast pumping.

The breast milk expression and storage bag system includes one or more breast milk expression and storage bag devices and an optional shell and may further include a vacuum source and tubing which connects the breast milk expression and storage bag device to the vacuum source and transmits cycles of suction from the vacuum source to the breast milk expression and storage bag device. The vacuum source may be a manual pump or an electric motor, for example, which may be a piston pump, for example, and may be powered through connection by a cord to a wall outlet and/or by batteries such as rechargeable batteries. The funnel portion of the breast milk expression and storage bag device is positioned against the breast and collects milk during a pumping session.

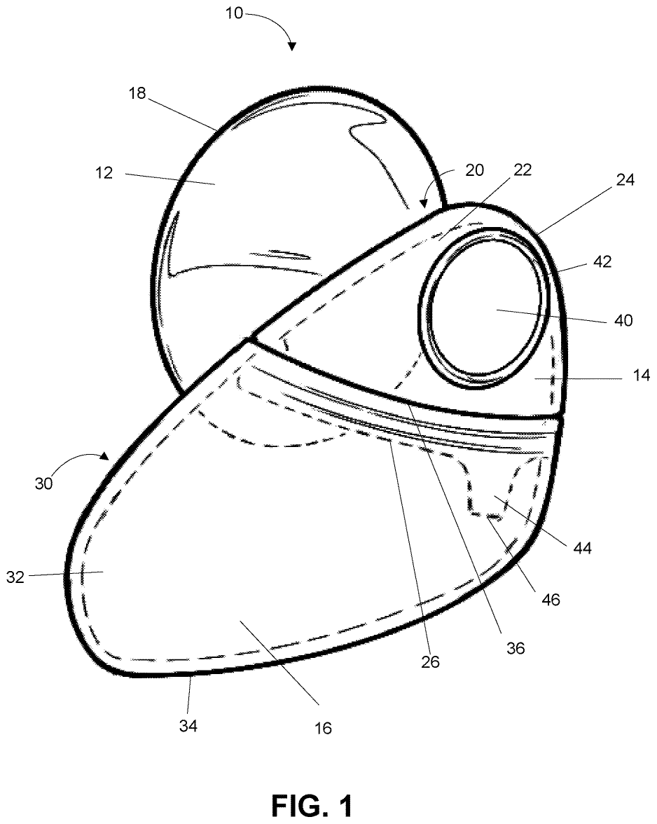

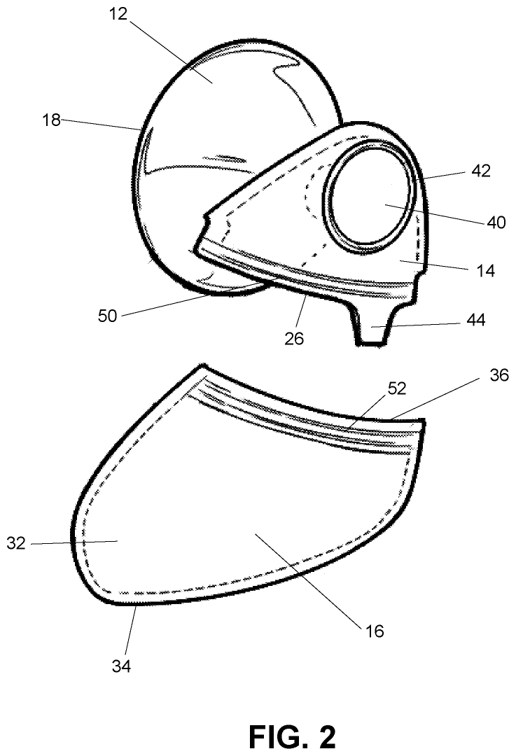

FIG. 1 shows a front perspective view of a breast milk expression and storage bag device 10 configured as a liner device according to various embodiments. The device 10 includes a funnel portion comprising a funnel liner 12, an upper portion 14, and a storage reservoir 16 as a lower portion. The device 10 may be made from a flexible sheet material such as food-grade, BPA-free low-density polyethylene (LDPE), polyethylene (PE), or polyethylene terephthalate (PET). In some embodiments, each of the funnel liner 12, upper portion 14, and storage reservoir 16 are made of the same flexible material, while in other embodiments, different materials may be used for one or more of these portions of the device 10. For example, in some alternative embodiments, the funnel portion of the device 10 may be a more rigid material, such as a more rigid material than the upper portion 14 and storage reservoir 16. In some such embodiments, the more rigid funnel portion may act as a funnel itself, without the need for a separate external rigid funnel to support it. In such embodiments, the funnel portion of the device 10 is not a funnel liner 12 rather is a funnel itself. However, in the embodiments shown herein, the funnel portion is a funnel liner 12 that is flexible and uses a separate external rigid funnel to support it.

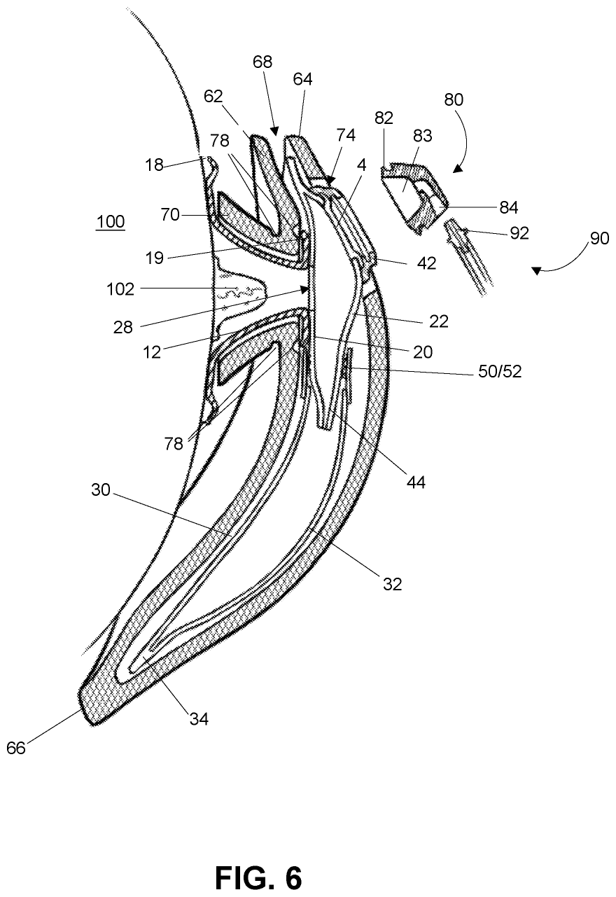

The funnel liner 12 is configured to extend within and fully line the interior of a more rigid external funnel which supports it. For example, the funnel liner 12 may extend beyond and somewhat around the rear edge of the external funnel, which may be provided by the funnel 70 of shell 60 or by a separate external funnel. In various embodiments, the external funnel may be provided by the shell 60, such as funnel 70 as described later herein. The outer shell 60 may also be coupled to the outer wall of the funnel liner 12. Alternatively the funnel liner may have structural rings coupled to the outer wall of the funnel liner to prevent the funnel portion from collapsing upon suction. However, the funnel may alternatively be a separate component of a stand alone component, such as the funnel of a traditional breast pump system. The funnel liner 12 may be tubular, open throughout and at each end, and generally conically shaped. The rear end 18 or edge may be the widest portion (largest diameter) of the funnel liner 12 and sized to extend beyond and/or around the wide end of the external funnel while narrowing as it extends forward surrounding the user's nipple. (However, in alternative embodiments, such as embodiments in which a diaphragm is located at the front of the funnel portion, the diameter of the front end of the funnel portion may widen and/or be wider than the rear end 18). The front end 19 or edge of the funnel liner 12 (see FIG. 6-8) connects to the rear wall 20 of the upper portion 14 of the device 10 around the entire circumference of the front end 19, to provide a liquid tight connection between these portions. In the example shown, the front end 19 flares outward to allow for a larger surface area of connection between the inner surface of the funnel liner 12 and the outside surface of the rear wall 20. While the funnel portion (whether a funnel liner 12 or non-liner self-supporting funnel portion) is generally a cohesive component of the breast milk expression and storage bag device 10, in alternative embodiments, the funnel portion may be detachable from the rest of the device 10. In such embodiments, the funnel portion may be reusable or may be disposable. It may attach to the rest of the device 10, such as to the upper portion 14, by a removable attachment method such as a Ziplock type tongue and groove connection, a hoop and loop connection, an adhesive connection, or other connection.

The upper portion 14 and storage reservoir 16 are removably connected together to form an interior space into which the milk is collected. They each include two sheets which form the front wall and the back wall, which have a liquid tight connection around their outer edges. The upper portion 14 includes a front wall 22 and a rear wall 20 (see FIGS. 6-8) which are connected together (and/or contiguous) around the entire outer edge 24, including the lower edge 26, except at the lower edge 46 of valve 44 which is open. Alternatively, outer edge 24 may also include one or more disconnected gaps to form apertures for liquid tight connection to a funnel portion and/or a diaphragm, for example.

The storage reservoir 16 similarly includes a front wall 32 and a rear wall 30 which are connected together in a liquid tight manner (and/or are contiguous) around the entire outer edge 34 except they are not connected along the upper edge 36. Rather, the upper edge 36 is open, allowing the lower edge 26 of the upper portion 14 to insert into the storage reservoir 16 at the upper edge 36 of the storage reservoir 16 as shown in FIG. 1.

In alternative embodiments, the upper portion 14 and/or the storage reservoir 16 may not be directly connected together around their outer edges but rather may be indirectly connected around a portion of or all of their outer edges. For example, they may include additional sheet material forming a liquid tight connecting bridge between the front and rear walls 22, 32, 20, 30 which may allow them to separate further apart, such as for expandability and greater storage capacity in the storage reservoir 16.

The upper portion 14 is connected to the storage reservoir 16 at or near the lower edge 26 and the upper edge 36. This connection may be a separable and/or reconnectable and resealable connection such that the storage reservoir 16 may be removed from the upper portion 14, such as after collecting milk in the storage reservoir 16. In other embodiments, the upper portion 14 and storage reservoir 16 may be two portions of a single continuous piece. For example, in a single piece embodiment, a single front sheet and a single rear sheet may be sealed together and/or contiguous around the entire outer edges, forming both the upper portion and lower portion. The single front sheet and single rear sheet may be brought together/connected to join the sheets, such as with a heat crimp, to separate the upper and lower portions and create an inner valve such as a tension valve. For example, a heat crimp could create an inner funnel of the same shape, and optionally a tear off portion could be located below the valve. A closure mechanism such as a Zip-Lock, Adhesive, Fold and Clip, etc. may be located before the tear off location for closure after removal. In other embodiments, the device 10 may include a skirt adaptation and/or a box base.

In the embodiment shown, the separable connection between the upper portion 14 and the storage reservoir 16 is a Ziplock type of connection including one or more male tracks (ribs) and/or one or more female tracks (grooves) for a mating connection. Other removable connections that may be used include perforations for disconnection by tearing, resealable connections such as hook and loop connections, adhesive, press to seal (such as GRIPTEX), or fold and clip (such as a reinforced edge with bendable end pieces extending outward, such that the device may be closed and wrapped around the reinforced edge, then held in the wrapped position by folding the bendable end pieced against the wrapped material), for example.

In the embodiment shown, the separable connection is also sealable, and may be used to seal the storage reservoir 16 closed along the upper edge 36 after detaching the storage reservoir 16 from the upper portion 14, making the storage reservoir 16 a liquid tight storage container after use during pumping. In this example, the connector 50 of the upper portion 14 may be located at or adjacent to the lower edge 26 and connects to a connector 52 of the storage reservoir 16 that may be located at or adjacent to the upper edge 36 of the storage reservoir 16. For example, the connector 50 may be less than 2 cm, or less than 1 cm above the lower edge 26 of the upper portion 14, such as between 1 or 2 or 3 mm and 1 cm, such as about 5 mm. The connector 50 may also be at least 1 mm or at least 2 mm or at least 3 mm above the lower edge 26. Likewise, the connector 54 may be less than about 2 cm, or less than about 1 cm from the upper edge 36 of the storage reservoir 16. The connector 52 may also be at least 1 mm or at least 2 mm or at least 3 mm below the upper edge 36, such as between 1 or 2 or 3 mm and 1 cm, such as about 5 mm. This may allow enough mater to peal back the upper edge 36 to grip onto it and detach it from the upper portion 14. This probably needs to be within 5 mm of the upper edge, but there still needs to be enough of an edge to peel it back and grab onto it to detach from the upper portion. After separation of connector 50 of the upper portion 14 from connector 52 of the storage reservoir 16, connector 52 of the front wall 32 may connect to the connector 52 of the rear wall 30 of the storage reservoir 16 to seal the storage reservoir 16 closed. As such, the connector 52 of the front wall 32 and the rear wall 30 may be configured to connect with each other (as well as their aligned counterpart connectors 50 in the upper portion 12). For example, if a mating type of connection is used, the connector 52 of the front wall 32 of the storage reservoir 16 (such as a male or female Ziplock connector) must be the opposite of the connector 52 of the rear wall 30 (such as a female or male connector) of the storage reservoir 16. Similarly, the connector 52 of the front wall 32 of the storage reservoir 16 must be the opposite of the connector 52 of the front wall 22 of the upper portion 14. Likewise, the connector 52 of the rear wall 30 of the storage reservoir 16 must be the opposite of the connector 50 of the rear wall 20 of the upper portion 14. As a result, the connector 50 of the front wall 22 of the upper portion 14 is the same type as the connector 52 of the rear wall 30 of the storage reservoir 16 (such as both male or both female types). Similarly, the connector 50 of the rear wall 20 of the upper portion 14 is the same type as the connector 52 of the front wall 32 of the storage reservoir 16 (such as both female or both male types). Various embodiments may attach in this way, with a ZIPLOCK type attachment, though other attachments may alternatively or additionally be used such as adhesive like peal and stick adhesives or press-to-seal adhesives.

In the embodiment shown, the upper portion 14 is slightly narrower at the lower edge 26 than the upper edge 36 of the storage reservoir 16. This allows the lower edge 26 of the upper portion 14 to be smoothly inserted into the storage reservoir 16 at upper edge 36 without bending or folding, to form a smooth and continuous, liquid and air tight connection at connectors 50 and 52. Thus, in this example, connector 50 is located on the outer surface of the upper portion 12, while connector 52 is located on an inner surface of lower portion 14. This allows for a continuous smoother outer edge perimeter formed by outer edges 24 and 34. However, in alternative embodiments, the upper portion 12 and storage reservoir 16 may be configured to connect with a different shape or in different ways. In this way, the form may taper to allow the upper portion 14 to fit into the storage reservoir 16. In various alternative shapes, the receiving mouth at the upper edge 36 storage reservoir 16 (32 with 50/52) may be slightly wider to receive the connection of the lower edge 26 of the upper portion 14. Furthermore, there may be a small taper or decrease in size at the outer edge 24 of the upper portion, where it inserts into the storage reservoir 16 allow for insertion into the storage reservoir 16.

The upper portion 14 includes additional features to allow for formation of a vacuum within the interior space. The rear wall 20 of the upper portion 14 includes an aperture 28, around which the funnel liner 12 (or alternatively the non-liner funnel portion) connects as described above. (See FIGS. 6-8). Aperture 28 allows for the passage of milk from the space within the funnel liner 12 (or non-liner funnel portion) into the space within the upper portion 14. The front wall 22 also includes a diaphragm 40 and a ring 42 surrounding the diaphragm 40 which are connected to and continuous with the front wall 22. In various embodiments, the ring 42 and diaphragm 40 are fixedly connected to the front wall 22 and are not removable. Rather, they form a portion of device 10 which may be purchased by consumers as a complete one piece unit for a single use. In other embodiments the diaphragm may be located elsewhere in the device, such as at or near the front end of the funnel portion, or in a wall of the storage reservoir 16. Regardless of its location, the diaphragm may be fully integrated with the device such that there is no passage for milk outside of the device through the diaphragm.

The diaphragm 40 may be made of a flexible and stretchable material such as food-grade, BPA-free low-density polyethylene (LDPE), polyethylene (PE), or polyethylene terephthalate (PET), TPU, or silicone. In some embodiments, the diaphragm 40 may be made from the same material as the upper portion 14 and/or the front wall 22 (and optionally the storage reservoir 16 and/or the funnel liner 12). In such embodiments, the diaphragm 40 may be continuous with the front wall 22 as a portion of the same sheet of material as the front wall 22, with the ring 42 applied and sealed onto the front wall 22 around the circumference of the diaphragm 40, such as on the outer surface of the front wall 22. In other embodiments, the diaphragm 40 may be a different material than the front wall 22, such as a more stretchable and elastic material than the front wall 22. In such embodiments, device 10 may be constructed by forming an aperture in the front wall 22 and sealing the diaphragm 40 into the aperture in an airtight manner around the circumference of the diaphragm 40. The ring 42 may be sealed to the front wall 22 and/or diaphragm 40 around the circumference of the diaphragm in an airtight manner, such as at the location of connection between the diaphragm 40 and the ring 42. These components may be attached together by heat welding or adhesive such as food safe adhesive, for example.

The ring 42 which surrounds the diaphragm 40 may be a more rigid material than the diaphragm 40. In addition to stabilizing the diaphragm 40 when vacuum is applied to it, ring 42 may also provide an airtight connection for formation of a vacuum air space, as will be described later in this disclosure.

The upper portion 14 further includes a valve 44. In the embodiment shown, the valve 44 is located at the lower edge 26 of the upper portion 14, below the connector 50. As shown in the dashed lined of FIG. 1 (to show the inner aspects of the connected device 10), the valve 44 is also below the connector 52 of the storage reservoir 16 when the upper and lower portions 14, 16 are connected. When vacuum is applied to the device 10 and transmitted into its interior space through the diaphragm 40, the valve 44 is sealed closed by the vacuum within the upper portion 14. In this way, the valve 44 prevents backflow of milk from the storage reservoir 16 into the upper portion 14. When there is no vacuum, valve 44 is open, allowing milk to flow downward through valve 44 at the opening at the lower edge 46 and into the storage reservoir 16 by gravity.

The valve 44 may be any type of one-way valve to allow passage of fluid only from the upper portion 14 to the storage reservoir 16 of the device 10, such as a duck bill valve. In some examples, the valve includes a front wall formed by a lower portion of the front wall 22 and a rear portion formed by a lower portion of the rear wall 20. The front wall and rear walls of the valve 44 may abut each other. As such, when moistened by milk, they may adhere together. This adhesion may be increased as they are drawn together by suction provided during the vacuum pulses in the upper portion 14. However, the adhesion may be loose enough such that the front and rear walls of the valve 44 separate when the suction is released the gravity pulls the milk downward. Alternatively, a valve such as a duck bill valve or a flapper valve, may be inserted and sealed into the upper portion 14 at or near the lower edge 26.

In various embodiments, the diaphragm 40 and ring 42 and/or the valve 44 may be in other locations within the upper portion 14 or within other portions of the device 10 such as within the funnel liner 12 (or funnel portion) or within the storage reservoir 16. Furthermore, in some embodiments, the upper portion 14 may not be a distinct portion but rather may be included in a front part of the funnel liner 12 (or funnel portion).

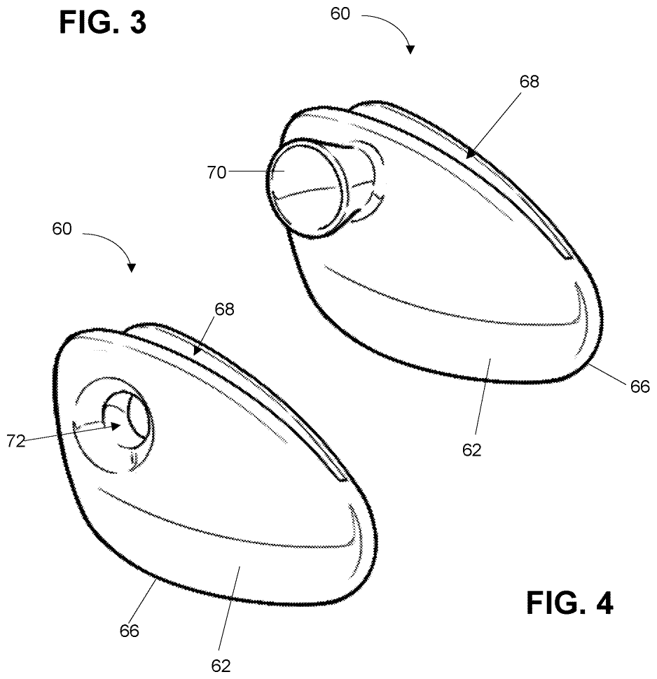

The device 10 may optionally be used with a shell, such as shell 60 shown in FIGS. 3-5. The shell 60 may provide a physical support for the flexible device 10 and may be sized and shaped to match and accommodate the device 10. The angle and shape of the shell may vary from that shown. For example, in some embodiments, the shell may have a less steep angle to lay flatter and more flush against the breast. In various embodiments, the material of the device 10 may be sufficiently rigid and strong to be used alone, without the shell 60, or to be used with only a rigid funnel piece and not an entire shell, such as a funnel that is sufficiently rigid to not collapse upon suction from vacuum pressure applied through the device by an externally applied vacuum source and/or to ensure stability during diaphragmatic oscillation. However, when a shell is used, such as shell 60, the device 10 may be inserted into the shell 60 before use and then may be used to collect milk while within the shell 60. In some embodiments, the shell 60 may be sized and shaped to insert into the cup of a bra, such as between the bra cup and the user's breast. Because it is relatively flat and thin, the shell 60 fits comfortably within the bra in a low profile manner which is not visibly apparent or is minimally and not excessively visibly apparent. The device 10 may be inserted into the shell 60 before or after then shell 60 is inserted into the bra. In various embodiments, the front and rear walls 64, 62 of the shell may be between about 1 mm and 5 mm thick, for example. The shell may be between about 3 mm and about 1 cm thick, with the device 10 inserted, before filling occurs. Other thicknesses may alternatively be used. The material of the shell, while somewhat rigid to provide support and prevent compression of the device 10, may be sufficiently flexible to be comfortable for the user. For example, the shell may be compressed EVA foam, TPU, Silicone, or other closed cell soft material, for example.

The shell 60 includes a front wall 64 and a rear wall 62 which are spaced apart by and connected together along a portion of the outer edge 66. The outer edge 66 connection may be rounded and smooth, as shown in this example. The space between the front wall 64 and rear wall 62 allows for insertion of the device 10 as well as expansion of the device 10 as it fills with milk and prevents compression of storage reservoir 16.

Opening 68 provides access to the space within the shell 60 for insertion and removal of the device 10. In the embodiment shown, opening 68 is located at and extends across a majority of the top edge and a portion of a side edge of the shell 60, allowing for easy access to the inner space and easy removal of the device 10 when full of milk, without compression of the device 10. In other embodiments the opening may be located along a side or bottom edge of the outer shell, and may be smaller or larger than the opening 68 shown in the example.

The rear wall 62 of the shell include the funnel 70. The funnel is conical, being wider at the rear edge than at the location of connection to the rear wall 62. In the embodiment shown, the funnel 70 is contiguous with the rear wall 62 and may be formed together of the same material. However, in other embodiments, the funnel 70 may be removably connected and reattachable to the shell 60, such as through insertion of the funnel 70 into an aperture in the rear wall 62 of the shell, for a screw fit or friction fit, for example. Removal of the funnel 70 from the rear wall 62 may be useful for storage, transportation, or cleaning. In this embodiment shown, the funnel 70 is connected to the rear wall 62 and may be inverted for storage and transportation. In FIG. 3, the funnel 70 projects outward and is in position for use during breast pumping. In FIG. 4, the funnel 70 has been inverted into the interior space of the shell 60. In such embodiments, the funnel material must be sufficiently flexible to allow for inversion, while also sufficiently rigid to support pressure against the breast to form an airtight seal during pumping. The shell 60 may further include relief channel at which the thickness of the shell material is narrowed relative to the adjacent material, at or near where the funnel adjoins the rear wall 62. One or more of such relief channels may be included enable the funnel 70 to bend more easily at these locations. In this way, the relief channels may allow for a planned deformation and flex in a planned way so that the funnel 70 flip in and out repeatedly and stay in position and/or flex to compress and then expand to snap into shape and position for use. An example of such relief channels are shown in the circumferential relief channels 78 which encircle the funnel 70 in FIGS. 6-8. However, in alternative embodiments, the funnel 70 may not be invertible but may only be available in the use configuration shown in FIG. 3, or may be a separate removable component.

The shell 60 further includes a front aperture 74 in the front wall 64 to accommodate passage of the vacuum components, as described further below.

The device 10 may releasably attach to or couple to the shell in one or more locations to provide additional stability to the device 10 when used as a liner, such as to stay in position when vacuum is applied to the inside of the device. For example, the device 10 may include connection features on an outer wall, such as raised connector points such as loops that connect to the funnel, raised ridges that slide into slots on the external funnel or are pinched to be held into place by external funnel, button like protrusions that go through slots on external funnel, rings around the outer wall that slide into slots on external funnel, tubes which may optionally be disposable that may be inserted device to provide structural support to keep its shape, and/or adhesive material on the outer wall of the liner.

In some embodiments, the external funnel may be comprised of a plurality of longitudinal sections, each section extending from the front to the back end of the funnel and forming a portion of the encircling wall of the funnel and thus having a rounded or arced shape matching the curvature of the external funnel tube. The external funnel may be divided into two, three, four or five section such longitudinal sections. When placed together into a funnel shape, the sections may be held together in a rigid funnel formation by rings at the front and/or back ends of the sections which connect to rings. The liner device may include a plurality of sleeves extending longitudinally along and connected to the outside of the funnel liner. The interior wall of the sleeves may be the funnel liner wall. The exterior wall of the sleeves may be one or more separate sheets which may be connected to the exterior wall of the sleeve by multiple lines of connection extending longitudinally along a length of the separate sheet(s), such as by multiple parallel lines and/or evenly spaced lines of connection such as heat welded lines of connection. The sleeves may extend the entire length of the funnel liner, from the front to the rear end, or only along a portion of the funnel liner. The sleeves may be open at each end (the front and rear ends) and may be sized and shaped to securely fit the funnel sections within them, and there may be a matching number of sleeves and funnel sections. As such, the funnel sections may be inserted into the sleeves in the appropriate orientation (e.g. with the curve inward and the front and rear ends oriented appropriately), with the ends of the sections extending beyond and through the ends of the sleeves. The section ends may then be connected to the rings to hold the funnel sections together as an assembled funnel, with the wall of the funnel liner extending within it. The rings which support and connect to the ends of the funnel sections may be adjustable to increase and decrease in diameter (such as by a telescoping formation) and/or may be provided as a part of the system in various sizes, to allow for the diameter or one or both ends of the funnel formed by the funnel sections to be adjustable smaller or larger as needed.

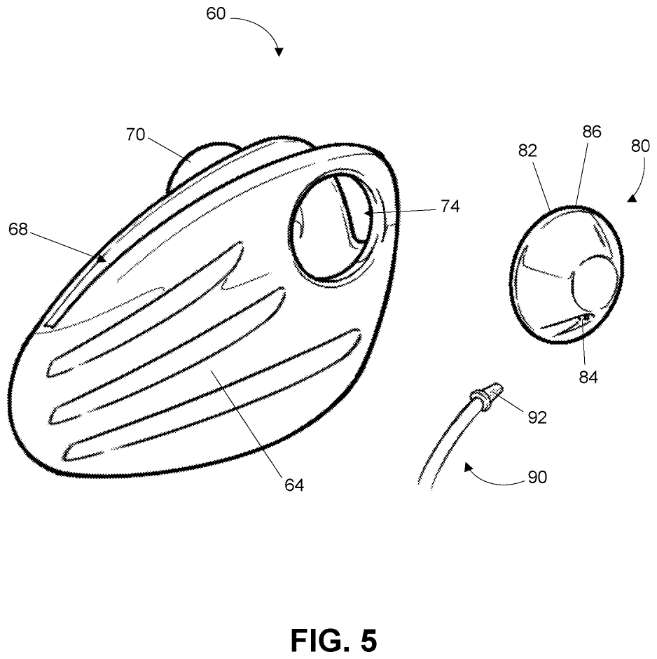

Whether or not the device 10 is used with a shell or alone, a cover may be attached to the diaphragm ring 42 on the outer surface of the device to create a vacuum space. Alternatively, the diaphragm portion of the device may include a cover (permanently or removably) connected to or integrally formed the outside of the device over the diaphragm to create a vacuum space, such as at connected to or formed contiguously with the diaphragm ring 42. In the embodiments shown, the vacuum space is created by the diaphragm cap 80, shown in FIG. 5. The diaphragm cap 80 is dome shaped, though other shapes are possible, provided the diaphragm cap 80 seals around the diaphragm 40 and provides an air space 83 for oscillation of the diaphragm 40, such as in response to an externally applied vacuum or manually, by which a vacuum is created within the funnel liner 12 (or self-supporting funnel portion). In the example shown, the diaphragm cap 80 is configured to connect to ring 42 at connector 82 and provides an air space 83 within the diaphragm cap 80. Diaphragm cap 80 further includes a port 84 for connection to vacuum tube 90. Vacuum tube 90 may be a flexible but semi rigid tube like those used with standard breast pump systems. Vacuum tube 90 connects to the vacuum cap 80 at a first end 92, and connects to a vacuum source (not shown) at the opposite second end (not shown). The vacuum source may provide intermittent vacuum pulses in the standard manner for breast pumps. The vacuum pulses are transmitted through the vacuum tube 90 and into the air space 83 of the vacuum cap 80 which acts as a vacuum space, providing suction to the diaphragm 40. The suction against the diaphragm 40 causes it to stretch outward, transmitting the vacuum to the interior space of the upper portion 14 and closing the valve 44. This intermittent vacuum suction stimulates release of milk which passes through the interior of the funnel liner 12 and into the upper bag portion 14, and then falls into the storage reservoir 16 when the vacuum is released and the valve 44 opens. In other embodiments, the diaphragm cap 80 may provide a space for the diaphragm to oscillate such as by manual or other direct physical manipulation without vacuum suction from a breast pump.

Although the diaphragm 40 and diaphragm ring 42 are shown as round, other shapes may be used in various embodiments, such as oval, square or polygonal. The diaphragm cap 80 may then have a corresponding shape to connect with the diaphragm ring 42.

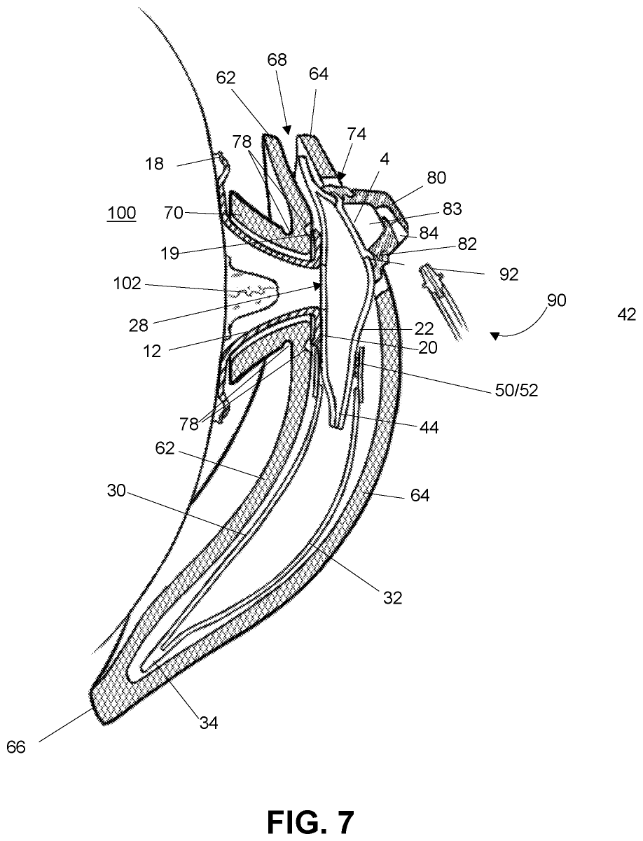

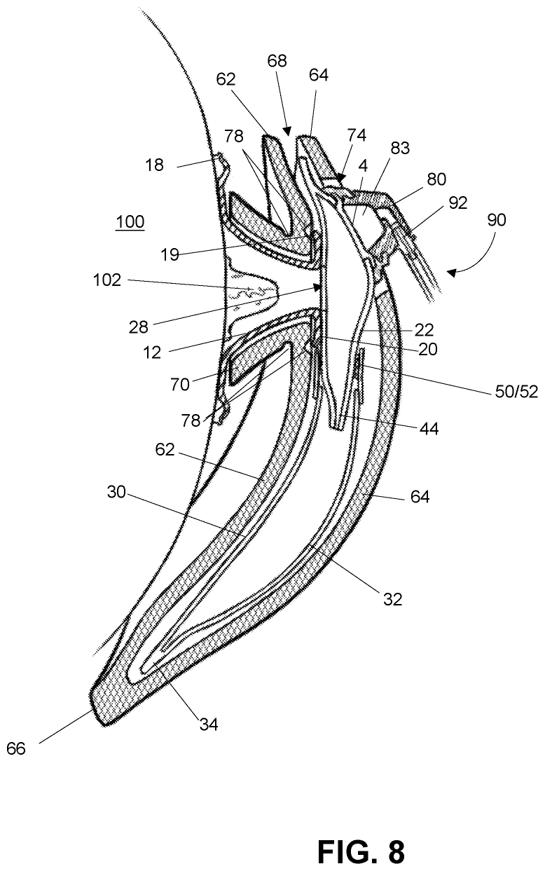

Cross sectional views of the device 10 within the shell 60 are shown in FIG. 6 8, depicting the system in use. In FIG. 6, the device 10 is present in the shell 60. The diaphragm 40 is aligned with the front aperture 74 of the shell 60, and the ring 42 project outward, extending partially through and beyond the front aperture 74. In this image, the diaphragm cap 80 and vacuum tube 90 are not connected to the breast milk expression and storage bag device 10. In FIG. 7, the vacuum cap 80 is connected to the ring 42 by a snap fit connection, while in FIG. 8, the vacuum tube 90 is connected to the vacuum cap 80. In use, the vacuum tube 90 may be attached to the vacuum cap 80 before or after connection to the device 10.

In the embodiment shown, the vacuum cap 80 includes connector 82 at the outer edge 86 which form a connection feature for connecting to matching connection features on the inner surface of the ring 42 above the diaphragm 40. In this example, the outer edge 86 includes a tongue and groove, which releasably connect to a matingly sized and shaped groove and tongue on the interior of the ring 42. More or fewer tongues and grooves may alternately be used. Furthermore, other types of connections may be used as the connection features, and the locations of the connection features may be adjusted. For example, the connection features of the ring 42 may alternatively or additionally be located on the outer or front surface of the ring 42 and/or the outer edge of the ring 42. Similarly the connection features of the vacuum cap 80 may alternatively or additionally be located on the rear surface and/or in edge of the vacuum cap 80. Other types of connections which may be used include screw connections, clip on, or quarter/partial turn lock, for example. Furthermore, some embodiments may optionally include a flexible gasket between the vacuum cap 80 and the ring 42. Regardless of the type of connection used for the connection features, the connection should be sufficiently airtight so as to form a vacuum space between the vacuum cap 80 and the diaphragm 40 to transmit suction into the upper portion 14 of the device 10.

The vacuum cap 80 further includes a port 84 for connection to the vacuum tube 90. The port 84 includes a connector for connection to the first end 92 of the vacuum tube 90 and may provide a tunnel opening 86, if necessary, extending between the port 84 and the air space 83 of the vacuum cap 80. The connector may be a screw on or press fit connection. a universal or brand-specific connecter may be used in various embodiments.

In the embodiment shown in FIG. 6-8, the funnel liner 12 is in place within the funnel 70 of the shell. The rear end 18 of the funnel liner 12 extends beyond and somewhat around the end of the funnel 70. In this way, in use, the rear end 18 of the funnel liner 12 is compressed between the funnel 70 and the surface of the breast 100 of the user, creating an airtight seal allowing for transmission of suction through the diaphragm 40 and into the interior of the upper portion 14 and the interior of the connected funnel liner 12. The nipple 102, within the interior of the funnel liner 12, is stimulated by the intermittent suction to release milk, which flows through the funnel liner and/or is secreted into the upper portion 14 of the device 10. As can be best appreciated in FIG. 6-8, the milk never contacts any portion of the system other than the device 10. In this way, the shell 60, vacuum cap 80, vacuum tube 90 and mechanical vacuum pump remain clean and complex cleaning routines are not needed. Rather, after use, the device 10 can be removed from the shell 60 (if used) and detached from the vacuum cap 80. The device 10 can then be used for storage of the milk, or the milk can be transferred to another container. The shell 60, vacuum cap 80, and vacuum tube 90 may be used again later for another pumping session without cleaning, since there was no exposure to milk during pumping.

As discussed above, the device 10 may serve as both a milk collection and storage device as well as a barrier to prevent contact between reusable components such as the optional shell, an optional separate funnel, the vacuum cap 80, the vacuum tube 90 and the milk. It further does not include any reusable vacuum transmission components such as a flange or diaphragm and does not require a back flow protector. The device 10 may be a single use, disposable component, or it could be reusable and washable, while the other components of the breast pump system may be permanent, reusable and non-disposable such as the vacuum cap 80, vacuum tube 90, vacuum pump and the shell 60. In various embodiments of the device 10 may alternatively be washable and reusable.

In various embodiments, when the device 10 is provided to a user (such as upon purchase), the front walls 22, 32 and rear walls 20, 30 of both the upper and lower portions 14, 16, or at least of the storage reservoir 16, may be in an abutting relationship, without a gap between them. In this way, as the device 10 fills with milk, and particularly the storage reservoir 16, the front walls 22, 32 and back walls 20,30 may separate only as much as needed to accommodate the milk. In this way, there may be no need to release displaced air and there may be no air vent. However, in some embodiments, the device 10 may include an air vent, such as a flexible valve, to release displaced air, which may be located at or near the top of the upper portion 14 to reduce the risk of milk leakage.

In use, once the funnel liner 12 is in position on the breast (such as within the shell 60 or a funnel piece, or without a supplementary support) and connected to the vacuum cap 80, vacuum tube 90 and mechanical vacuum source, vacuum pulses may be started. For example, the motor of the mechanical vacuum source may be activated to begin vacuum pulses, or vacuum pulses may be applied manually. The diaphragm 40 may be pulled outward/forward with each vacuum pulse, transmitting the vacuum as a pressure drop in the interior of the upper portion 14 and funnel liner 12, creating suction which pulls on the nipple 102 to encourage milk secretion and also closes the valve 44. When the vacuum pulse is released, the vacuum pull on the nipple 102 is released and the valve opens 44, allowing milk to flow into the storage reservoir 16.

After pumping is finished, the motor is turned off (or the manual pumping is stopped) and the funnel liner 12 in the funnel (or funnel portion without external funnel) is removed from the breast. The storage reservoir 16 may be disconnected from the upper portion 14 and/or the funnel liner 12 (or self-supporting funnel portion) may then be sealed shut with a liquid tight closure using connector 52 to seal the front wall 32 to the rear wall 30. The removed portions may be discarded, and the milk may be transported and stored in the separated and sealed storage reservoir 16. Alternatively, if the funnel liner 12 (or self-supporting funnel portion) and/or upper portion 14 are not removed and/or are not removable, these portions may be collapsed, rolled up and/or folded against the filled and sealed storage reservoir and remain attached while occupying minimal volume during milk transportation and storage. The milk may then be stored within the reservoir portion, and may optionally be refrigerated or frozen within the reservoir portion for longer term storage. The milk may then later be fed to a child after storage.

Using the device 10 as disclosed herein, the milk only contacts the inside of the device 10 and never contacts other components such as the reusable components. There is also no risk of contact between milk and the vacuum tube 90 or the vacuum system. Furthermore, there is no permanent diaphragm or valve at risk of contacting the milk or requiring washing or sterilization, as in other systems. As such, the other components remain clean and do not require washing or sterilization between pumping sessions. A user may, therefore, proceed with one or more subsequent pumping sessions by inserting a new device 10 into the shell 60, for example, and pumping the milk as described above.

Although only a single device 10, outer shell 60, vacuum cap 80, and vacuum tube 90 are shown, the breast pump system may optionally include two breast milk expression and storage bag device 10, two vacuum caps 80, optionally two shells 60, such as those shown and/or described herein and which may be identical and/or opposite and/or symmetrical to each other, as well as two vacuum tubes 90, to connect each vacuum cap 80 to the motor or manual pump or pumps as the vacuum source. When the breast pump system includes two shells 60, two breast milk expression and storage bag device 10, two vacuum caps 80, and two vacuum tubes 90, both breasts may be pumped simultaneously.

Various embodiments of the device 10 may be manufactured using methodologies such as blow molded film or extruded film with secondary heat welding. For example, for two parts being created, a film that may be placed into a heated mold and air may be blown into. For the upper portion 14, air may be blowing air in through the valve opening 46. For the storage reservoir 16, air may be blowing in through the open upper edge 36. This may pre-form the upper and lower portions 14, 16 into pillowed shapes and may allow for a better single material use of the upper portion 16. Alternatively, the upper portion front and rear walls 22, 20 and lower portion front and rear walls 32, 20 may be created by bringing two separate films together on a roller, then heat stamp, seal, and cut along the sealed outer edges at once. Funnel liner 12 may be attached to the upper portion 14 by a heat weld or adhesive process, for example. The funnel liner may be formed as a cone from a single sheet with a single seem, or may be formed from two sheets with two seams, or multiple sheets with multiple seams, and may be heat melded or blow molded into shape, for example. The funnel liner 12 may be attached to the upper portion 14 (or optionally elsewhere) in a liquid tight manner by heat welding or adhesive, for example. In some embodiments, after all portions are formed as separate parts, the upper portion 14 may be connected to the storage reservoir 16 by the ZIPLOCK or other connection means.

The use of thin flexible sheets or films of material for the breast milk expression and storage bag device not only aids in manufacturing but also in filling with milk (without the need for an external funnel or support such as a shell, as described above) and in packaging, transportation and storage of the devices for users. The breast milk expression and storage bag devices may be provided to users as a single, complete integrated device that includes a funnel portion, a diaphragm, a valve, and a storage reservoir, and optionally an upper portion, as a substantially or completely flat and thin device in which all components are packed flat, such as in a stacked arrangement in a multidevice container such as a box, and ready for immediate use. The flat breast milk expression and storage bag device may optionally also include a diaphragm cap attached to the device in the ready to use form and disposable/single use like the device 10, or the diaphragm cap may be separate and optionally reusable.

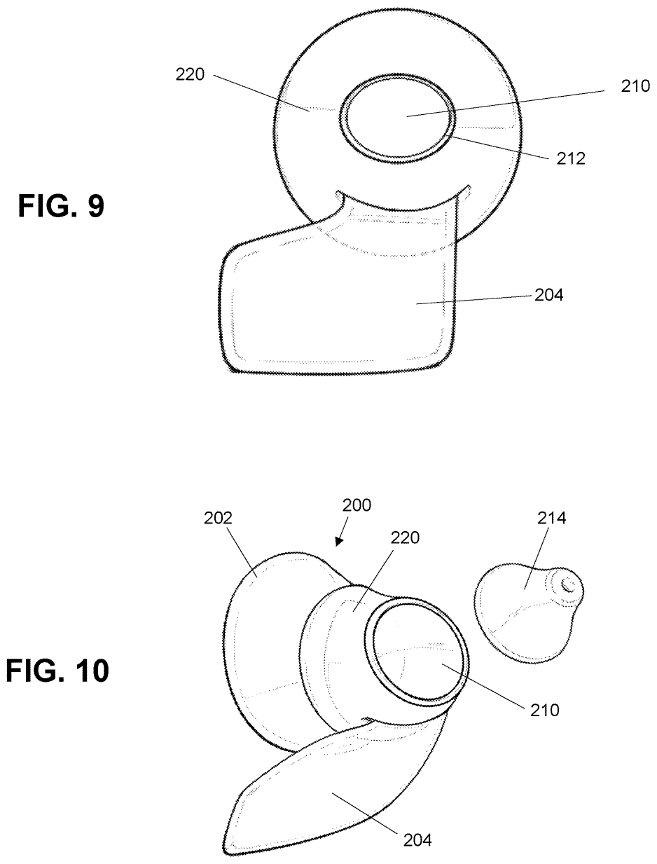

Alternative embodiments of the device are shown in FIG. 9-10. In this embodiment, the device 200 includes a funnel liner 202 and a single chamber 204 for milk collection and storage. The device 200 further includes a diaphragm 210 in a diaphragm ring 212 configured to connect with a cap 214 to form a vacuum air space and connect with a vacuum source, as described with regard to device 100. As in the above embodiments, the device may be fully constructed from a thin film material, with the possible exception of the diaphragm and diaphragm ring. In this embodiment, the device 200 is supported by and fully lines rigid support 220.

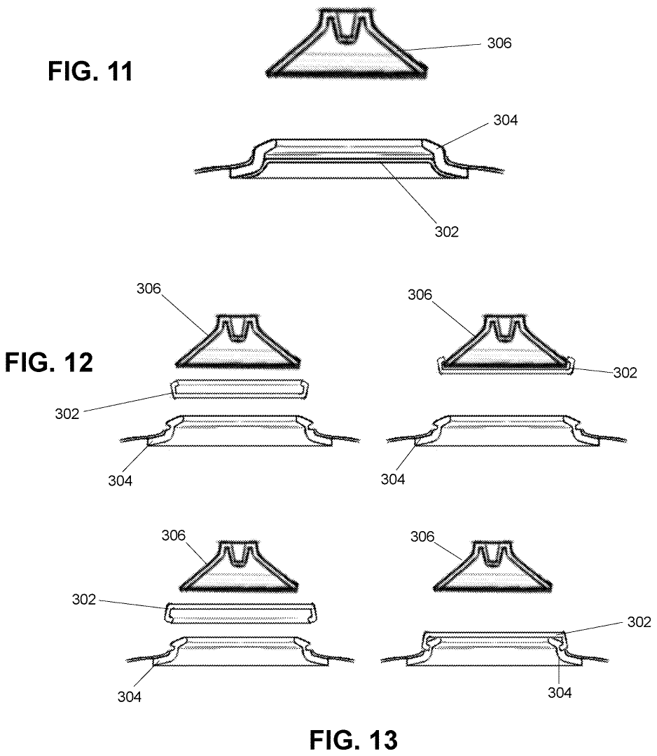

Alternative embodiments of the diaphragm and diaphragm cap are shown in FIGS. 11-13. These may alternatively be used in any of the devices described above. In some embodiments, the diaphragm 302 may be co-molded with the diaphragm ring 304, such as for retention of the diaphragm cap 306, and may be preassembled and non-removable. An example of this is shown in FIG. 11. In some embodiments, the diaphragm 302 may be molded separately as a cap to be placed in the assembly prior to user use, and may be optionally removed, such as prior to disposal, such as for single-sort recycling. An example of this is shown in FIGS. 12 and 13. In some embodiments, the diaphragm 302 may be like a cap that goes over the diaphragm cap 306, able to be reused for each item, with the device utilizing a one-way valve for air draw or a removable section to remain food-safe by the user. An example of this is shown in FIG. 12. The diaphragm 302 is shown between the diaphragm ring 304 and the diaphragm cap 306, and may go up into the diaphragm cap 306, flexing to wrap around it and creating an additional seal against the wall of the bag.

In various embodiments, the thickness of the diaphragm ring (as measured perpendicular to the device wall, for example), may be small in order to keep the device flat and thin when stacked and/or folded and for ease of transportation and packaging. For example, the diaphragm ring may be between about 0.5 and about 10 mm thick, or between about 0.5 mm and about 8 mm, or between about 0.75 and about 8 mm, or between about 0.75 and about 6 mm, or between about 3 and about 6 mm. The thickness (height) may be equal throughout, or may vary, and these values may represent the thickness (height) at the most thick location, for example. The diaphragm itself may add additional thickness at the location of the ring, where it is bonded to the ring, such as about 0.5 mm to about 10 mm, or about 0.5 mm to about 8 mm, or about 1 to about 5 mm, or about 2 to about 4 mm.

In the foregoing description, the inventions have been described with reference to specific embodiments. However, it may be understood that various modifications and changes may be made without departing from the scope of the inventions. The disclosure is not intended to limit the inventions to the embodiments disclosed and many modifications and variations are possible in view to the above teachings. For example, various embodiments may be combined, and reference to one embodiment or some embodiments does not preclude one or more methods, functions, steps, features, results, or hardware implementations of different embodiments from being combined.

What is claimed is:

1. A bag device for breast milk expression and storage, the bag device comprising:
a storage reservoir configured to store the breast milk after expression;
a funnel portion comprising one or more outer walls forming a tubular shape which tapers to a more narrow diameter as it extends toward a front end and an interior space within the one or more walls, the funnel portion connected directly or indirectly to the storage reservoir;
a flexible diaphragm, wherein the flexible diaphragm is configured to transmit externally applied vacuum pulses into the interior air space of the funnel portion as suction, wherein the flexible diaphragm is connected to an outer wall of the device in a liquid tight manner; and
a valve configured to allow breast milk flow into the storage reservoir and prevent backflow of breast milk out of the storage reservoir.

2. The device of claim 1 wherein the storage reservoir, the funnel portion, the flexible diaphragm, and the valve are fully integrated as a single device.

3. The device of claim 1 further comprising an upper portion connecting the funnel portion and the storage reservoir.

4. The device of claim 3 wherein the flexible diaphragm is located in a wall of the upper portion.

5. The device of claim 4 wherein the valve is located between the upper portion and the storage reservoir, such that breast milk located in the upper portion flows through the valve and into the storage reservoir by gravity.

6. The device of claim 4 wherein the upper portion and the storage reservoir are releasably connected by a connection feature to allow separation of the reservoir portion from the upper portion.

7. The device of claim 6 wherein the connection feature is configured to allow liquid tight closure of the reservoir portion after separation of the reservoir portion.

8. The device of claim 1 wherein the funnel portion comprises a flexible liner configured to line an interior wall of a separate more rigid external funnel, wherein the funnel portion prevents contact between the breast milk and the external funnel.

9. The device of claim 1 wherein the flexible diaphragm is within the funnel portion.

10. The device of claim 1 wherein the flexible diaphragm is located within and integrated with the storage reservoir.

11. The device of claim 1 further comprising:
a ring surrounding the flexible diaphragm; and
a cap connected to or configured to connect to the ring on an exterior aspect of the system and forming an airspace between the flexible diaphragm and the cap, the cap further configured to connect to a vacuum tube.

12. A breast milk expression and storage bag device comprising:
a funnel portion comprising one or more walls forming a tubular shape having an open rear end and tapering inward as it extends toward a front end, the funnel portion configured to surround an interior space;
an upper portion comprising:
one or more outer walls comprising flexible sheets configured to surround an interior space;
a flexible diaphragm integrated with the one or more outer walls of the upper portion;
an aperture in the one or more outer walls of the upper portion, the front end of the funnel portion connected to the upper portion at the aperture of the upper portion to provide a liquid tight connection between the interior space of the funnel portion and the interior space of the upper portion;
a storage reservoir connected to the upper portion, the storage reservoir comprising one or more walls comprising flexible sheets configured to surround an interior space; and
a valve configured to provide a sole flow path for one direction flow of breast milk from the interior space of the upper portion to the interior space of the lower portion.

13. The device of claim 12 wherein the storage reservoir is located below the upper portion.

14. The device of claim 12 wherein the valve is located at or formed by a lower edge of one or more of the one or more walls of the upper portion.

15. The device of claim 12 further comprising a sealable closure providing liquid tight enclosure of the storage reservoir.

16. The device of claim 15 wherein the upper portion and the storage reservoir are releasably connected to each other by the sealable closure.

17. The device of claim 12 further comprising a ring surrounding the flexible diaphragm, the ring comprising a more rigid material than a material of the flexible diaphragm.

18. The device of claim 17 wherein the material of the flexible diaphragm comprises the same material as a material of the one or more outer walls with which it is integrated.

19. The device of claim 12, further comprising an outer shell, wherein the outer shell comprises a front wall, a rear wall and a space between the front and rear walls, wherein the device is sized and shaped to fit within the outer shell space when empty and when filled with milk.

20. A method of expressing and collecting breast milk that eliminates the need to clean pump parts after a pumping session comprising:
connecting a single-use disposable breast pump part set comprising a funnel, a valve, a diaphragm, and a milk reservoir to a breast pump;
abutting the rear end of the funnel portion to the breast while ensuring the nipple is inserted into the interior space of the funnel portion;
operating the breast pump to create diaphragmatic oscillation of the diaphragm to extract milk from the nipple that flows into the funnel, through the valve, and down into the milk reservoir;
completing the pumping session, and then;
removing the single-use pump part set from the breast and nipple;
disconnecting the pump part set from the breast pump; and
disposing of the pump part set after use, eliminating the necessity of cleaning parts after using them.
